(19) United States
(12) Patent Application Publication
(10) Pub. No.: US 2025/0163947 A1
(43) Pub. Date: May 22, 2025

(54) TELESCOPING POLE ASSEMBLY

(71) Applicant: TECHNOLOGIES CGC INC., Quebec (CA)

(72) Inventors: Carl BOUCHARD, Quebec (CA); Bruno DESAULNIERS, Quebec (CA); Richard LABBE, Quebec (CA)

(21) Appl. No.: 18/841,837

(22) PCT Filed: Mar. 16, 2023
(86) PCT No.: PCT/CA2023/050336

§ 371 Date: Aug. 27, 2024

Related U.S. Application Data
(60) Provisional application No. 63/320,406, filed on Mar. 16, 2022.

(51) Int. Cl. F16B 7/14; F16B 9/00

(52) U.S. Cl. CPC F16B 7/1427; F16B 9/05; F16B 2200/406

(57) ABSTRACT

A telescoping pole assembly includes first and second pole segments and a dampening assembly having a bushing, a gasket and a spigot. The first pole segment has a closed distal end and an open proximal end in which a distal end of the second pole segment is received. The bushing is attached to the distal end of the second pole segment, has a neck extending therebeyond. The gasket defines gasket opening and is shaped to fill an inner area of the first pole segment. The spigot extends from a gasket face towards the distal end of the second pole segment. In response to the pole segments moving towards each other, the neck contacts the gasket face and at least partially obstructs the gasket opening. In response to the pole segments moving away from each other, the neck is separated from the gasket face and does not obstruct the gasket opening.

### TECHNICAL FIELD

The present technology relates to telescopic pole assemblies.

### BACKGROUND

Telescopic pole assemblies are commonly used in a variety of fields, and include one or more inner pole segments that are received in one or more outer pole segments.

Conventionally, telescopic pole assemblies are not provided with damping assemblies. As a result, an inner pole segment, when released from a connection to the outer pole segment, and when subjected to gravitational forces, can move fast relative to an outer pole segment. This can result in the inner pole segment damaging the outer pole segment or vice versa and/or causing loud noises, can cause equipment damage or even hurt a nearby person.

Some telescopic pole assemblies may be provided with damping assemblies such as air gaskets to dampen downward movements of the inner pole segment, but these can create a vacuum effect when the inner pole segment is moved away from the outer pole segment, which can result in poor usability of the telescopic pole assembly. Some telescopic pole assemblies can have check valves which can be expensive to manufacture and can draw in liquids through external apertures, which can make cleaning difficult.

Therefore, there is a desire for a telescopic pole assembly that can overcome at least some of the above-described drawbacks.

### SUMMARY

It is an object of the present technology to ameliorate at least some of the inconveniences present in the prior art.

According to one aspect of the present technology, there is provided a telescoping pole assembly includes a first pole segment, a second pole segment and a damping assembly. The first pole segment has a distal end which is closed and a proximal end which is open. The second pole segment has a distal end received in the proximal end of the first pole segment. The first and second pole segments are moveable towards and away from each other along a longitudinal axis. The damping assembly includes a bushing, a gasket and a spigot. The bushing, which is attached to the distal end of the second pole segment, has a neck extending beyond the distal end of the second pole segment towards the distal end of the first pole segment. The bushing defines a bushing channel therein. The gasket, which is sized and shaped to fill an inner area of the first pole segment, has a gasket opening defined therein. The spigot extends from a gasket face towards the distal end of the second pole segment. A free end of the spigot is received in the bushing channel. In response to the first and second pole segments moving towards each other, the neck contacts the gasket face and at least partially obstructs the gasket opening. In response to the first and second pole segments moving away from each other, the neck is separated from the gasket face and does not obstruct the gasket opening.

In some embodiments, the free end of the spigot has a lip, and the bushing has a step, abutment of the lip and the step delimiting movement of the spigot in the bushing channel.

In some embodiments, the second pole segment is solid.

In some embodiments, the spigot is attached to the gasket by a fastener extending through the gasket and into the spigot.

In some embodiments, the telescoping pole assembly further includes a locking system configured to selectively lock a relative position of the first and second pole segments.

In some embodiments, the locking system comprises a sleeve extending around a proximal end of the first pole segment as well as the second pole segment, the sleeve having sleeve threads on an inner face thereof and the proximal end of the first pole segment having pole threads which can engage with the sleeve threads.

In some embodiments, a portion of the sleeve in contact with the second pole segment has a smaller perimeter than a portion of the sleeve in contact with the first pole diameter.

In some embodiments, the locking system comprises clips configured to selectively fix a relative position of the first pole segment and the second pole segment.

In some embodiments, the telescoping pole assembly further includes at least one hook attached to the proximal end of the second pole segment.

In some embodiments, the first pole segment is connectable to a support.

In some embodiments, the support is a mobile structure.

In some embodiments, the gasket is made of resilient material.

In some embodiments, the first and second pole segments have a cylindrical configuration.

In some embodiments, in a cross-section taken across a horizontal plane passing through the first pole segment, the second pole segment and the bushing, the first pole segment defines an inner pole diameter, the second pole segment defines an outer pole diameter that is smaller than the inner pole diameter of the first pole segment, and the bushing defines an outer bushing diameter that is smaller than the inner pole diameter of the first pole segment and an inner bushing diameter that is smaller than the outer pole diameter of the second pole segment.

### Advantages

In certain embodiments, the damping assembly can dampen the second pole segment as it moves toward the first pole segment.

In certain embodiments, the damping assembly is configured to stop the damping effect when the first and second pole segments move away from the one another such that the damping assembly does not cause a vacuum effect.

In certain embodiments, the damping assembly is fully integrated within the telescopic pole assembly, thereby minimizing outer apertures.

In certain embodiments, the damping assembly is manufactured in a cost-efficient way.

In the context of the present specification, unless expressly provided otherwise, the words “first”, “second”, “third”, etc. have been used as adjectives only for the purpose of allowing for distinction between the nouns that they modify from one another, and not for the purpose of describing any particular relationship between those nouns.

It must be noted that, as used in this specification and the appended claims, the singular form “a”, “an” and “the” include plural referents unless the context clearly dictates otherwise.

As used herein, the term “about” in the context of a given value or range refers to a value or range that is within 20%, preferably within 10%, and more preferably within 5% of the given value or range.

As used herein, the term “and/or” is to be taken as specific disclosure of each of the two specified features or components with or without the other. For example “A and/or B” is to be taken as specific disclosure of each of (i) A, (ii) B and (iii) A and B, just as if each is set out individually herein.

Implementations of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects, and advantages of implementations of the present technology will become apparent from the following description, the accompanying drawings, and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 is a perspective view taken from a top, front, right side of a telescopic pole assembly having an inner pole segment, an outer pole segment and a damping assembly, according to certain embodiments of the present technology.

FIG. 2A is a cross-sectional view of the telescopic pole assembly of FIG. 1 showing the damping assembly responsive to the inner pole segment being moved toward the outer pole segment.

FIG. 2B is a close-up of the damping assembly of FIG. 2A.

FIG. 3A is a cross-sectional view of the telescopic pole assembly of FIG. 1 showing the damping assembly responsive to the inner pole segment being moved away from the outer pole segment.

FIG. 3B is a close-up of the damping assembly of FIG. 3A.

FIG. 4 is a perspective view taken from a top, front, right side of the telescopic pole assembly of FIG. 1 connected to a mobile structure.

### DETAILED DESCRIPTION

The present disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including”, “comprising”, or “having”, “containing”, “involving” and variations thereof herein, is meant to encompass the items listed thereafter as well as, optionally, additional items. In the following description, the same numerical references refer to similar elements.

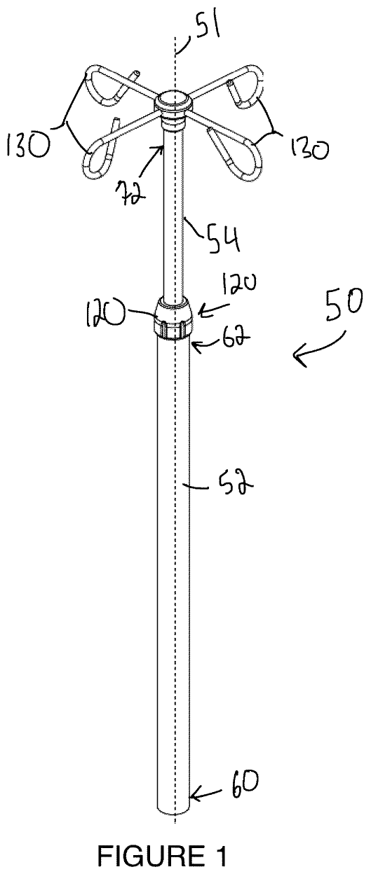

Referring to FIGS. 1, 2A, 2B, 3A and 3B, there is provided a telescoping pole assembly 50 according to certain embodiments of the present technology. Uses of the telescoping pole assembly 50 are not particularly limited. For example, as described and illustrated herein, the telescoping pole assembly 50 can be used to support intra-venous bags at adjustable heights thereof. In such uses, the telescoping pole assembly 50 is supported vertically to a support, such as a mobile structure, a wheelchair, a bed. However, other uses of the telescoping pole assembly 50 are also within the scope of the present technology, in which the telescoping pole assembly 50 can be supported and used horizontally or at any required inclination.

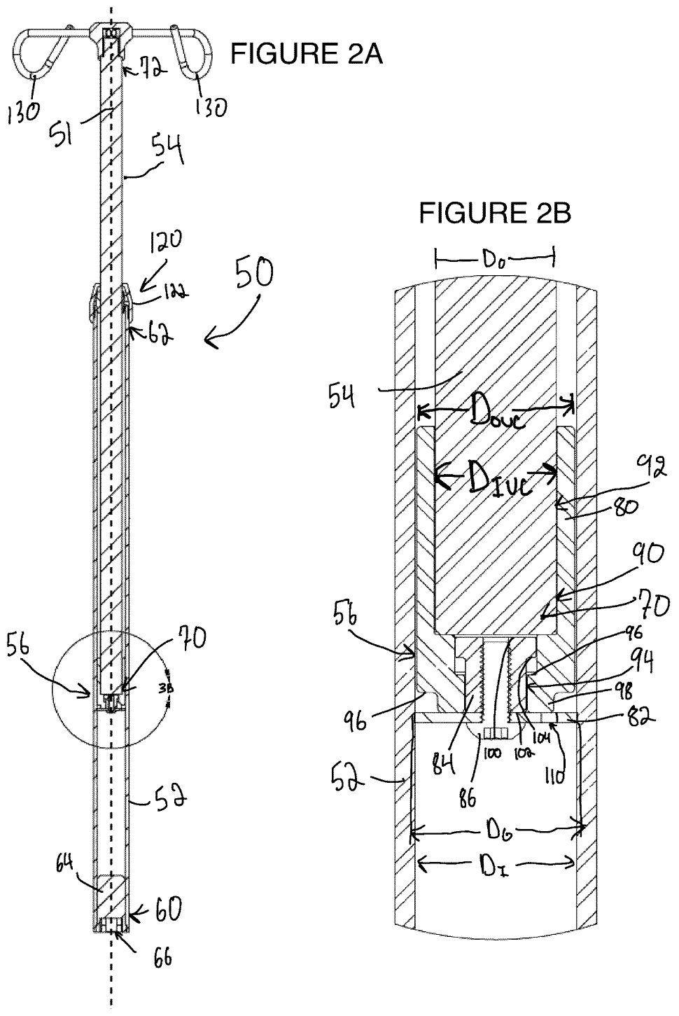

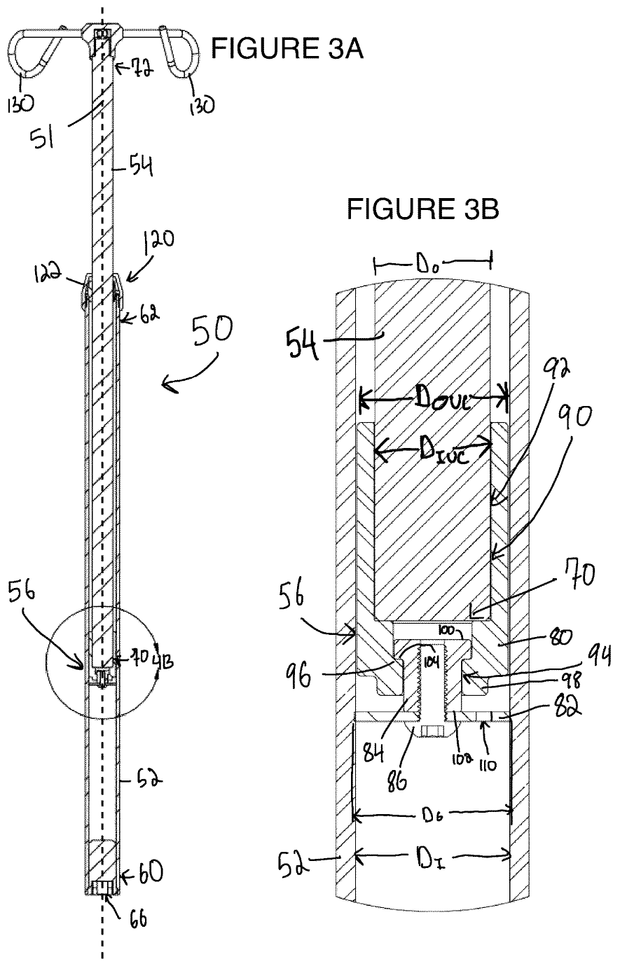

The telescoping pole assembly 50 includes an outer pole segment 52, an inner pole segment 54 that is at least partially received in the outer pole segment 52 and a damping assembly 56 (best seen in FIGS. 2B and 3B). As will be described in greater detail below, the inner pole segment 54 is moveable relative to the outer pole segment 52 along a longitudinal axis 51 that extends vertically and that passes through centers of the outer and inner pole segments 52, 54. The outer and inner pole segments 52, 54 have a cylindrical configuration. In other words, cross-sections of the outer and inner pole segments 52, 54 taken along a horizontal plane generally define circles. It is contemplated that in other embodiments, the outer and inner pole segments 52, 54 could have another configuration such as, for example, a rectangular configuration in which the cross-sections of the outer and inner pole segments 52, 54 taken along a horizontal plane generally define rectangles.

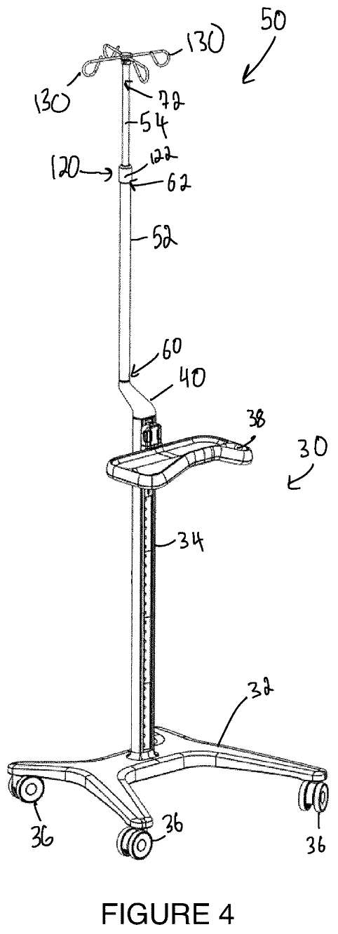

With reference to FIGS. 1, 2A, 2B, 3A and 3B, the outer pole segment 52 will first be described in greater detail. The outer pole segment 52, which has a distal end 60 and a proximal end 62, is tubular (i.e., hollow) along an entire length thereof. It is contemplated that in some embodiments, a portion of the outer pole segment could be solid (i.e., not hollow). The distal end 60 is closed by a sealing member 64. More specifically, the sealing member 64 hermetically seals the distal end 60 of the outer pole segment 52. In some embodiments, the distal end 60 could be generally airtight. Thus, air generally cannot enter or exit the outer pole segment 52 through the distal end 60. The sealing member 64 is made of a polymeric material such as rubber, although other materials such as metal are contemplated. The sealing member 64 defines a recess 66 for receiving a connector or a portion of a support for supporting the telescoping pole assembly 50 in a vertical orientation. An example support will be described later with reference to FIG. 4. On the other end, the proximal end 62 is open and receives part of the inner pole segment 54 therein. The outer pole segment 52 defines an inner diameter D_{I} that is generally uniform along the entire length thereof. It is contemplated that in other embodiments the inner diameter D_{I} could vary along the length of the outer pole segment 52.

It is contemplated that in some embodiments, at least a portion of the outer pole segment 52 could be solid (i.e., not hollow). In such embodiments, the sealing member 64 could be omitted.

The inner pole segment 54 will now be described in greater detail. The inner pole segment 54, which has a distal end 70 and a proximal end 72, is solid (i.e., not hollow). As such, the distal and proximal ends 70, 72 are closed. It is contemplated, however, that in some embodiments, at least a portion of the inner pole segment 54 could be hollow. The distal end 70 is received in the proximal end 62 of the outer pole segment 52 and is connected to a bushing 80 of the damping assembly 56. The inner pole segment 54 defines an outer diameter D_{O} that is generally uniform along the entire length thereof. It is contemplated that in some embodiments, the outer diameter D_{O} could vary along some of the length of the inner pole segment 54. The outer diameter D_{O} of the inner pole segment 54 is smaller than the inner diameter D_{I} of the outer pole segment 52, thereby enabling the inner pole segment 54 to be received in the outer pole segment 52.

With particular reference to FIGS. 2B and 3B, the damping assembly 56, which includes the bushing 80, a gasket 82, a spigot 84 and a fastener 86, will now be described in greater detail.

The bushing 80 is attached to the distal end 70 of the inner pole segment 54 and is received within the outer pole segment 52. Since the bushing 80 is configured to be received in the outer pole segment 52, the bushing 80 defines an outer diameter D_{OUC} that is smaller than the inner diameter D_{I} of the outer pole segment 52, such that the bushing 80 and the outer pole segment 52 have a clearance fit. Other fits are contemplated. The clearance fit between the bushing 80 and the outer pole segment 52 enables passage of air therethrough. In some embodiments, the bushing 80 could be configured to having a sliding fit with the inner diameter D_{I} of the outer pole segment 52, and the bushing could define one or more airways therethrough so that airflow is not impeded by the bushing 80.

The bushing 80 defines a bushing channel 90 having an upper channel section 92 and a lower channel section 94. The upper channel section 92 is configured to receive the distal end 70 of the inner pole segment 54. The upper channel 90 has an inner diameter D_{IUC} that is smaller than the outer diameter D_{O} of the inner pole segment 54, such that the bushing 80 and the inner pole segment 54 are connected by an interference fit. Other fits are contemplated. It is further contemplated that in other embodiments, the bushing 80 could be attached to the distal end 70 of the inner pole segment 54 differently. For example, the bushing 80 could be attached to the distal end 70 by an adhesive or by an airtight threaded connection. The lower channel section 94, which is configured to receive a free end 100 of the spigot 84, has a step 96 that, as will be described in greater detail below, is configured to delimit movement of the spigot 84 in the lower channel section 92. In some embodiments, the bushing channel 90 could be two distinct channels instead of having upper and lower channel sections 92, 94. In some embodiments, the bushing 80 may not extend along the distal end 70 of the inner pole segment 54 and may therefore define only a single channel, lower channel section 94.

The lower channel section 94 defines a neck 98. The neck 98 extends beyond the distal end 70 of the inner pole segment 54 towards the distal end 60 of the outer pole segment 52. The neck 98 has a diameter that is smaller than the outer diameter D_{OUC} of the bushing 80. As will be described below, the neck 98 can selectively contact the gasket 82.

The spigot 84, which is partially received in the lower channel section 94, extends from a gasket-engaging end 102 towards the free end 100. The free end 100 has a lip 104 that is engageable to the step 96 of the bushing 90. The spigot 84 defines a central aperture that extends from the gasket engaging end 102 towards the free end 100 for receiving the fastener 86 therein. As will be described below, it is contemplated that in some embodiments, the central aperture could be omitted.

The gasket 82 is sized and shaped to generally fill an inner area of the outer pole segment 52. More precisely, in the present embodiment, the gasket 82 defines a diameter D_{G} that is a greater that the inner diameter D_{I} of the outer pole segment 52, such that there is an interference fit between the gasket 82 and the inner diameter D_{I} of the outer pole segment 52. Other fits are contemplated. The gasket 82 is made of a resiliently deformable material such as rubber or other polymer so that when the inner pole segment 54 and hence the gasket 82 moves relative to the outer pole segment 52, the gasket 82 deforms instead of getting blocked. Different dimensions and material combinations of the gasket 82 are within the scope of the present technology, and may be chosen to optimize a seal and/or minimize friction between edges of the gasket 82 and the outer pole segment 52 whilst permitting relative movement between the inner and outer pole segments 54, 52. The gasket 82 defines a central aperture for receiving the fastener 86 and a gasket opening 110. The gasket opening 110 is offset from the central aperture, and is positioned to be partially below the neck 98 of the bushing 90. It is contemplated that in some embodiments, the gasket 82 could define additional gasket openings. The gasket 82 is attached to the spigot 84 (i.e., fixedly connected) by the fastener 86. More precisely, the fastener 86 extends through the central apertures of the gasket 82 and the spigot 84, such that the gasket-engaging end 102 of the spigot 84 engages the gasket 82. It is contemplated that in some embodiments, the gasket 82 could be attached to the spigot 84 differently. For instance, the gasket 82 could be attached to the spigot 84 by an adhesive. In such embodiments, the fastener 86, the central aperture of the gasket 82 and the aperture of the spigot 84 could be omitted.

The gasket 82, the spigot 84 and the fastener 86 are generally fixed relative to one another, but are moveable relative to the bushing 80. As will be described in greater detail below, when the inner pole segment 54 is moving in a downward direction (i.e., towards the outer pole segment 52), the bushing 80 moves downwardly relative to the gasket 82, the spigot 84 and the fastener 86 until the neck 98 contacts the gasket 82. In the present embodiment, the neck 98 engaging the gasket 82 coincides with the free end 100 of the spigot 84 abutting the distal end 70 of the inner pole segment 54. It is contemplated that the neck 98 could contact the gasket 82 without the free end 100 abutting the distal end 70 of the inner pole segment 54. Since, as mentioned above, the damping assembly 65 is configured so that the gasket opening 110 is partially below the neck 98, when the neck 98 contacts the gasket 82, the neck 98 partially obstructs the gasket opening 110 such that the damping assembly 56 dampens the downward motion of the inner pole segment 54. On the other hand, when the inner pole segment 54 is moving in an upward direction (i.e., away from the outer pole segment 52), the inner pole segment 54 and the bushing 80 move upwardly relative to the gasket 82, the spigot 84 and the fastener 86 until the lip 104 of the spigot 84 abuts the step 96, at which point the gasket 82, the spigot 84 and the fastener 86 move along with the bushing 80. In this configuration, the neck 98 does not obstruct the gasket opening 110, such that the damping assembly 56 does not create a vacuum or otherwise pressure decrease and thus does not cause a suction effect during the upward motion of the inner pole segment 54.

Referring to FIGS. 1, 2A and 3A, the telescoping pole assembly 50 also includes a locking system 120 configured to selectively lock a relative position of the inner and outer pole segments 52, 54. The locking system 120 includes a sleeve 122 that extends around the proximal end 62 of the outer pole segment 52 and around a portion of the inner pole segment 54. Given the difference in diameters of the inner and outer pole segments 52, 54, a lower portion of the sleeve 122, which is in contact with the outer pole segment 52, has a smaller perimeter than an upper portion of the sleeve 122, which is in contact with the inner pole segment 54. The sleeve 122 has threads on an inner face thereof that can engage with sleeve threads on the proximal end 62 of the outer pole segment 52. Fastening the sleeve 122 to the proximal end 62 of the outer pole segment 52 results in compressing the inner and outer segments 52, 54, thereby locking a relative position of the inner and outer pole segments 52, 54. In some embodiments, the locking system 120 may comprise a locking plug (not shown) provided between the sleeve 122 and the inner pole segment 54. The locking plug, without hermetically sealing the proximal end 62, can help stabilize the inner pole segment 54 relative to the outer pole segment. In some embodiments, the locking system 120 could include clips (not shown) configured to selectively fix a relative position of the inner and outer pole segments 52, 54.

The telescoping pole assembly 50 also includes four intravenous (IV) hooks 130 connected to the proximal end 72 of the inner pole segment 54. It is contemplated that more or fewer IV hooks 130 could be connected to the inner pole segment 54. In other embodiments, other types connectors could be connected to the telescoping pole assembly 50.

In the present embodiment, the telescoping pole assembly 50 is connected to a mobile structure 30, which includes a wheeled base 32 and a support 34.

The wheeled base 32, which generally forms an H-shape, has four wheels 36. It is contemplated that in some embodiments, the wheeled base 32 could have more or fewer than four wheels. The configuration of the wheeled base 32 can aid in providing stability to the mobile structure 32. Additionally, the configuration of the wheeled base 32 can assist in minimizing chances of feet of a user bumping into the wheel based 32. The support 34 extends generally vertically from the wheeled base 32. The support 34 has a tray 38 connected thereto, though it is understood that the tray 38 could be omitted in some embodiments. In other embodiments, the support 34 could have other components connected thereto. In the present embodiment, the support 34 is a mounting assembly as described in U.S. Provisional Patent Application No. 63/319,945, filed Mar. 15, 2021, the contents of which are incorporated herein by reference in their entirety. It is understood that in some embodiments, the support 34 could be a straight vertical pole. In other embodiments, the support 34 could be omitted and the telescoping pole assembly 50 could be connected directly to the wheeled base 32.

The telescoping pole assembly 50 is connected to the support 34 via a connector 40 that extends angularly relative to the support 34. It is contemplated that in some embodiments, the connector 40 could be configured to extend parallel or perpendicular to the support 34.

The damping assembly 56 in use will now be described.

When an item such as a saline bag or an IV pump is hung on one of the IV hooks 130, and the locking system 120 locking the relative position of the outer and inner pole segments 52, 54 is selectively unlocked, gravity causes the inner pole segment 54 to move in a downward direction. The damping assembly 56 dampens the downward motion of the inner pole segment 54, thanks to air dampening. More precisely, as the inner pole segment 54 moves in a downward direction (i.e., towards the outer pole segment 52), the bushing 80, which is fixed to the distal end 70 of the inner pole segment 54, also moves in a downward direction. However, since the gasket 82, the spigot 84 and the fastener 86 are moveable relative to the bushing 80, the gasket 82, the spigot 84 and the fastener 86 remain fixed relative to the outer pole segment 52 (i.e., don't move), in part due to the interference fit between the gasket 82 and the inner surface of the outer pole segment 52, until the neck 98 contacts the gasket 82. As described above, the neck 98 engaging the gasket 82 results in partially obstructing the gasket opening 110. Thus, since the outer pole segment 52 is closed at the distal end 60, air present within the outer pole segment 52 can only exit therefrom by passing through the partially obstructed gasket opening 110. Thus, when the outer pole segment 52 moves downwardly, there is an increase in air pressure within the outer pole segment 52, which dampens the motion of the inner pole segment 52.

On the other hand, when the inner pole segment 54 is moved in an upward direction (i.e., away from the outer pole segment 52), the bushing 80, which is fixed to distal end 70 of the inner pole segment 54, also moves in an upward direction. However, since the gasket 82, the spigot 84 and the fastener 86 are moveable relative to the bushing 80, the gasket 82, the spigot 84 and the fastener 86 remain fixed relative to the outer pole segment 52 (i.e., don't move), in part due to the interference fit between the gasket 82 and the inner surface of the outer pole segment 52. Thus, the neck 98 is separated from the gasket 82, and therefore does not obstruct the gasket opening 110. The gasket 82, the spigot 84 and the fastener 86 remain fixed relative to the outer pole segment 52 until the lip 104 abuts the step 96, at which point the gasket 82, the spigot 84 and the fastener 86 move along with the bushing 80 and the inner pole segment 54. As a result of the neck 98 not obstructing the gasket opening 110, air within the outer pole segment 52 can exit therefrom through the unobstructed gasket opening 110, such that the damping assembly 56 does not create a vacuum (i.e., does not cause a suction effect). Therefore, the inner pole segment 54 can be moved in an upward direction with little resistance.

Modifications and improvements to the above-described embodiments of the present invention may become apparent to those skilled in the art. The foregoing description is intended to be exemplary rather than limiting. The scope of the present invention is therefore intended to be limited solely by the appended claims.

What is claimed is:

1. A telescoping pole assembly comprising:
a first pole segment having a distal end which is closed and a proximal end which is open;
a second pole segment having a distal end received in the proximal end of the first pole segment, the first and second pole segments being moveable towards and away from each other along a longitudinal axis;
a damping assembly including:
a bushing attached to the distal end of the second pole segment, the bushing having a neck extending beyond the distal end of the second pole segment towards the distal end of the first pole segment, and the bushing defining a bushing channel therein;
a gasket sized and shaped to fill an inner area of the first pole segment and having a gasket opening defined therein;
a spigot extending from a gasket face towards the distal end of the second pole segment, a free end of the spigot being received in the bushing channel such that:
in response to the first and second pole segments moving towards each other, the neck contacts the gasket face and at least partially obstructs the gasket opening, and
in response to the first and second pole segments moving away from each other, the neck is separated from the gasket face and does not obstruct the gasket opening.

2. The telescoping pole assembly of claim 1, wherein the free end of the spigot has a lip, and the bushing has a step, abutment of the lip and the step delimiting movement of the spigot in the bushing channel.

3. The telescoping pole assembly of any one of claims 1 to 2, wherein the second pole segment is solid.

4. The telescoping pole assembly of any one of claims 1 to 3, wherein the spigot is attached to the gasket by a fastener extending through the gasket and into the spigot.

5. The telescoping pole assembly of any one of claims 1 to 4, further comprising a locking system configured to selectively lock a relative position of the first and second pole segments.

6. The telescoping pole assembly of claim 5, wherein the locking system comprises a sleeve extending around a proximal end of the first pole segment as well as the second pole segment, the sleeve having sleeve threads on an inner face thereof and the proximal end of the first pole segment having pole threads which can engage with the sleeve threads.

7. The telescoping pole assembly of claim 6, wherein a portion of the sleeve in contact with the second pole segment has a smaller perimeter than a portion of the sleeve in contact with the first pole diameter.

8. The telescoping pole assembly of claim 5, wherein the locking system comprises clips configured to selectively fix a relative position of the first pole segment and the second pole segment.

9. The telescoping pole assembly of any one of claims 1 to 8, further comprising at least one hook attached to the proximal end of the second pole segment.

10. The telescoping pole assembly of any one of claims 1 to 9, wherein the first pole segment is connectable to a support.

11. The telescoping pole assembly of claim 10, wherein the support is a mobile structure.

12. The telescoping pole assembly of any one of claims 1 to 11, wherein the gasket is made of resilient material. 13 The telescoping pole assembly of any one of claims 1 to 12, wherein the first and second pole segments have a cylindrical configuration.

14. The telescoping pole assembly of claim 13, wherein in a cross-section taken across a horizontal plane passing through the first pole segment, the second pole segment and the bushing,
the first pole segment defines an inner pole diameter;
the second pole segment defines an outer pole diameter that is smaller than the inner pole diameter of the first pole segment; and
the bushing defines an outer bushing diameter that is smaller than the inner pole diameter of the first pole segment and an inner bushing diameter that is smaller than the outer pole diameter of the second pole segment.
